(19) United States
(12) Patent Application Publication
(10) Pub. No.: US 2008/0076123 A1
(43) Pub. Date: Mar. 27, 2008

(54) Polymerase variants for DNA sequencing

(75) Inventor: Philip Richard Buzby, Brockton, MA (US)

Correspondence Address: COOLEY GODWARD KRONISH LLP;ATTN: Patent Group, Suite 1100, 777 - 6th Street, NW, WASHINGTON, DC, 20001, US

(73) Assignee: Helicos BioSciences Corporation, Cambridge, MA (US)

(21) Appl. No.: 11/528,959

(22) Filed: Sep. 27, 2006

(51) Int. Cl. C12Q 1/68; C12N 9/14

(52) U.S. Cl. 435/6; 435/194; 435/193

(57) ABSTRACT

Described herein is a mutant polymerase, specifically the Klenow exo⁻ polymerase in which the proline at amino acid position 680 is replaced by a glycine (P680G), and its use in sequencing a single polynucleotide template using single molecule sequencing.

### BACKGROUND

Electrophoresis-based methods of nucleic acid sequencing, such as those based on methods described by Sanger et al. (1977) PNAS 74:5463-5467, require multiple copies of the template polynucleotide being sequenced. Those methods also require substantial amounts of time and reagents. Many of those limitations are overcome using single molecule sequencing, as described by, for example, by Braslavsky, et al. 100 PNAS 39060-64 (2003). One variation of single molecule sequencing includes exposing a nucleic acid primer to a polynucleotide template in the presence of a polymerase and at least one labeled nucleotide capable of hybridizing with the template nucleotide downstream of the hybridized primer. As each labeled nucleotide is incorporated into the growing complement strand, the label is detected and its position on the template is noted. The sequence of the polynucleotide template is thereby determined.

Single molecule sequencing allows the sequencing of nucleic acids at an increased resolution and with an increased sensitivity as compared to conventional methods of sequencing. However, there is a continuing desire for improved sequencing methods that result in long read lengths.

### SUMMARY

The invention provides methods for increasing the accuracy of single molecule sequencing. The approach uses a polymerase that features reduced processivity in relation to a wild-type polymerase. The invention also provides a polymerase variant that selectively adds a single nucleotide in a template-dependent manner per addition cycle as described in detail below.

In one aspect, the invention provides methods for determining the sequence of a single polynucleotide strand or multiple single strands that are individually optically resolvable on a surface. In preferred methods a nucleic acid template, primer, or both is (are) attached to a surface. The resulting surface-bound template/primer duplex is exposed to a Klenow Fragment with reduced exonuclease activity in which a glycine is substituted for the proline at position 680 in the wild-type enzyme and a first labeled nucleotide under conditions that permit template-dependent nucleotide incorporation into the primer. After removing unincorporated nucleotide, signal is detected from incorporated nucleotide. The nucleotide addition cycle is repeated and the detection of signal from sequentially incorporated nucleotides permits determination of the sequence of the template.

In a preferred embodiment, primer nucleic acids are immobilized on a surface, such that at least some of them are individually-optically resolvable. A preferred surface is an epoxide-coated glass surface. The surface optionally can be passivated in order to reduce or eliminate non-specific signal (background). Terminal nucleotides (5′) on the template can be aminated in order to facilitate surface attachment. Templates are prepared by any available methods that generates fragments from about 10 to about 150 nucleotides in length. The 3′ terminus of the templates contains a sequence that hybridizes to the primer. In one embodiment, the primers are of like sequence and the complement of that sequence is attached to the 3′ end of the template (e.g., by ligation or enzymatically using, for example, terminal transferase).

Preferred methods of the invention comprise the use of an altered DNA polymerase in order to increase read-length in a single molecule sequencing reaction comprising multiple base addition cycles. A base addition cycle comprises the enzyme-catalyzed addition to template-bound duplex of a labeled nucleotide in a template-dependent manner, followed by removal of unincorporated nucleotide, detection of label attached to incorporated nucleotide, and removal (or optionally neutralization) of the label. Optional additional steps include rendering unreactive any residual atoms (i.e., “stubs”) left after removal of the label, the addition of oxygen scavenger if an optically-detectable label is used in order to improve resolution of the label.

Preferred methods of the invention utilize an optically-detectable label, such as a fluorophore. The cyanin-3 flour is a highly-preferred label or others as described below. Detection of incorporated nucleotide, via the label, is best accomplished using a light microscope equipped with a total internal reflection (tir) objective. Total internal reflection fluorescence is a well-characterized way of increase the light that actually reaches a substrate, thus resulting in the highest-intensity observation conditions.

In one aspect, methods for analyzing the sequence of a polynucleotide comprise contacting a nucleic acid duplex comprising a template and primer with a Klenow exo− DNA polymerase having a glycine for proline substitution at position 680 in the presence of a first labeled nucleotide under conditions that permit template-dependent incorporation of the labeled nucleotide; detecting any incorporated nucleotide; thereby determining the identity of the complementary base that served as a template in the polynucleotide; (d) optionally repeating steps (b)-(c) one or more times using a further single labeled nucleotide, to thereby determine the sequence of a polynucleotide based upon an order of incorporated nucleotides.

Other aspects and advantages of the invention are provided in the detailed description that follows.

### DETAILED DESCRIPTION

The invention provides improved methods for single molecule nucleic acid sequencing. According to the invention, template-dependent sequencing-by-synthesis is conducted on substrate-bound nucleic acid duplex using a polymerase having reduced exonuclease activity and reduced processivity relative to the wild-type, such as a Klenow exo− DNA polymerase having a glycine for proline substitution at position 680.

One aspect described herein relates to the development of a non-processive mutant polymerase, specifically the Klenow exo⁻ DNA polymerase in which the proline at amino acid position 680 is replaced by a glycine (P680G), for use in sequencing a single polynucleotide template using single molecule sequencing. A mutant Klenow exo polymerase having a P680G mutation was described by Tuske et al. (2000) JBC 275(31):23759-23768, which is incorporated herein by reference.

“Non-processive polymerases,” as used herein include polymerases mutated to minimize both 5′-3′ exonucloease activity and 3′-5′ exonucleoase activity relative to a corresponding wild-type polymerase and that possesses a higher affinity for a labeled nucleotide than for a primer nucleic acid. The P680G polymerase mutant is an example of a non-processive polymerase.

One particular advantage of a polymerase, such as the P680G in single molecule sequencing is that it provides increased accuracy in sequencing templates that contain a stretch of 2 or more bases of the same type, e.g., such as AA or AAA or GGGG or CCCCC. The increased accuracy resulting from the use of a polymerase with reduced processivity ensures that the growing complement strand accurately reflects the sequence of the template strand even in stretches of 2 or more identical bases.

Methods of the invention comprise sequencing a polynucleotide using a mutant Klenow exo⁻ polymerase having a P680G mutation. The detection and analysis of a first base addition before the addition of a second base allows for improved accuracy and sensitivity in sequencing.

In one aspect, described herein are methods for determining the sequence of a single polynucleotide molecule to which a nucleotide primer is hybridized comprising contacting the polynucleotide molecule with a Klenow exo⁻ DNA polymerase having a P680G mutation in the presence of a first labeled nucleotide under conditions that permit the polymerase to attach a single labeled nucleotide to the nucleotide primer when the nucleotide is complementary to the template, and detecting a signal from the attached labeled nucleotide. The detection of a signal from an attached labeled nucleotide permits determination of the sequence of the polynucleotide.

The methods and compositions described herein can be utilized in a wide variety of sequence related applications, including for example, identifying PCR amplicons, RNA fingerprinting, differential display, single-strand conformation polymorphism detection, dideoxy finger printing, restriction maps and restriction fragment length polymorphisms, DNA fingerprinting, genotyping, mutation detection, oligonucleotide ligation assay, sequence specific amplifications, for diagnostics, forensics, identification, developmental biology, molecular medicine, toxicology, and animal breeding.

Polynucleotides templates: Methods disclosed herein can be used to analyze the sequence of any polynucleotide, whether synthetic or derived from a natural source. The polynucleotide typically is one strand of a double stranded duplex, but can also be double stranded with single stranded regions. Synthetic polynucleotides include cDNA, which is complementary to at least part of an RNA molecule or transcript, regardless of whether the RNA molecule or transcript was produced in vivo or in vitro. The RNA transcript may result from the transcription of mutated genes, such as those found in cancerous or precancerous cells, in birth defects, or single nucleotide polymorphisms, and may include splice variants of transcripts.

Any polynucleotide molecule, naturally derived or synthetically made, can be sequenced by the methods described herein, including genomic DNA or RNA purified a prokaryote, eukaryote, plant, virus, fungus, bacteria, pathogenic organism, animal, mammal, dog, cat, sheep, cattle, swine, goat and human, or an isolated cell thereof. Any polynucleotide molecule obtained directly from an organism or from a biological sample obtained from an organism, e.g., from blood, urine, cerebrospinal fluid, seminal fluid, saliva, sputum, stool, body fluid and tissue can be sequenced by the methods described herein. The sequencing of these DNA or RNA molecules may permit the identification of pathogens and infectious agents, as well as genetic polymorphisms. Methods for preparing and isolating various forms of cellular nucleic acids are known. (See, e.g., Guide to Molecular Cloning Techniques, eds. Berger and Kimmel, Academic Press, New York, N.Y., 1987; Molecular Cloning: A Laboratory Manual, 2nd Ed., eds. Sambrook, Fritsch and Maniatis, Cold Spring Harbor Press, Cold Spring Harbor, N.Y., 1989.) The methods disclosed in the cited references are exemplary only and any variation known in the art may be used.

Nucleic acids, or portions thereof, prepared by various amplification techniques, such as polymerase chain reaction amplification, can also be sequenced by the methods described herein. (See U.S. Pat. Nos. 4,683,195, 4,683,202 and 4,800,159). However, one significant advantage of single molecule sequencing is that amplification is not necessary. Nucleic acids to be sequenced can alternatively be cloned in standard vectors, such as plasmids, cosmids, BACs (bacterial artificial chromosomes) or YACs (yeast artificial chromosomes). (See, e.g., Berger and Kimmel, 1987; Sambrook et al., 1989.) Nucleic acid inserts can be isolated from vector DNA, for example, by excision with appropriate restriction endonucleases, followed by agarose gel electrophoresis. Methods for isolation of insert nucleic acids are well known.

Primers: Primers are constructed so that at least a portion is complementary to a template sequence. In a preferred embodiment, primers are synthetic homoploymer sequences that match a complementary homopolymer sequence attached (e.g., enzymatically, ligated) to a terminal of the template. Generally, primers are between ten and twenty bases in length, although longer primers may be employed. In certain embodiments, the primer is from about 10 bases in length to about 100 bases in length. In certain embodiments, primers are designed to be exactly complementary in sequence to a known portion of a template polynucleotide. Known primer sequences can be used, for example, where primers are selected for identifying sequence variants adjacent to known sequences, such as where an unknown nucleic acid sequence is inserted into a vector of known sequence, or where a native nucleic acid has been sequenced partially. Methods for synthesis of primers of any given sequence are known, and automated oligonucleotide synthesizers are commercially available. [See, e.g., Applied Biosystems, Foster City, Calif.; Millipore Corp., Bedford, Mass.

Other embodiments involve sequencing a nucleic acid using the methods described herein in the absence of a known primer-binding site. In such cases, it may be possible to use random primers, such as random hexamers or random oligomers of 7, 8, 9, 10, 11, 12, 13, 14, 15 bases or greater in length, to initiate polymerization.

In some embodiments, multiple primers which are complementary along different portions of the same polynucleotide template can be employed.

Substrate:. Solid supports to which the template polynucleotide may be attached include, but are not limited to, supports that comprise glass, e.g., controlled pore glass (CPG), fused silica, epoxy, plastic, such as polystyrene (low crosslinked and high cross linked polystyrene), polycarbonate, polypropylene and poly(methymethacrylate), acrylic copolymer, polyamide, silicon, metal (e.g., alkanethiolate-derivatized gold), cellulose, latex, dextran, metal, nylon, gel matrix (e.g., silica gel) or composites. The surface of the substrate or support may be planar, curved, pointed, or any suitable two-dimensional or three-dimensional geometry. Suitable three dimensional substrates include, for example, spheres, microparticles, beads, membranes, slides, plates, micromachined chips, tubes, microwells, microfluidic devices, channels, filters, or any other structure suitable for anchoring a nucleic acid.

Nucleic acid template polynucleotides are attached to the surface such that the template/primer duplexes are individually optically resolvable. In one embodiment, a substrate is coated to allow optimum optical processing and nucleic acid attachment. The surface of a quartz slide is chemically treated to specifically anchor DNA templates while preventing nonspecific binding of free nucleotides. Substrates for use in the methods described herein can also be treated to reduce background. In one embodiment, the surface of the solid support is coated with epoxide or a derivitized epoxide. The surface can also be improved to improve the positioning of attached template/primer duplexes. As such, the surface can be treated with one or more charge layers (e.g., a negative charge) to repel a charged molecule (e.g., a negatively charged labeled nucleotide ). For example, a substrate can be treated with polyallyamine followed by acrylic acid to form a polyelectrolyte multilayer. The carboxyl groups of the polyacrylic layer are negatively charged and thus repel negatively charged labeled nucleotides, improving the positioning of the label for detection.

Various methods can be used to anchor or immobilize the polynucleotide template molecule to the surface of the substrate. The immobilization can be achieved through direct or indirect bonding to the surface. The bonding can be by covalent linkage. See, e.g., Joos et al., Analytical Biochemistry 247:96-101, (1997); Oroskar et al., Clin. Chem. 42:1547-1555, 1996; and Khandjian, Mol. Bio. Rep. 11:107-115, 1986. A preferred attachment is the direct amine bonding of a terminal nucleotide of the polynucleotide template to an epoxide integrated on the surface. Preferably, epoxide-coated glass surfaces are used for the direct amine attachment of template polynucleotides. Alternatively, the attachment can be achieved by anchoring a hydrophobic chain in a lipid monolayer or bilayer. Other methods known in the art for attaching nucleic acid molecules to substrates can also be used.

In one embodiment, one or more polynucleotide templates are hybridized to a fluorescently labeled primer and bound to the surface at a surface density low enough to resolve single molecules. The primed templates are detected through their fluorescent tags, their locations are recorded for future reference, and the tags are photobleached. The hybridization of one or more primers to a polynucleotide is well known in the art, see Sambrook and Russell, Molecular Cloning, A Laboratory Manual, Cold Spring Harbor Press, 3d ed. (2001) (“Sambrook and Russell”); Sambrook, Fritsch, and Maniatis, Molecular Cloning, A Laboratory Manual, Cold Spring Harbor Press, 2d ed. (1989) (“Sambrook et al.”); Ausbel et al., Current Protocols in Molecular Biology, John Wiley & Sons, Inc. (1995, including supplements through the August 2003) (“Ausbel et al.”).

Exo⁻ Klenow Fragment P680G: A polymerase used in methods described herein is the Exo⁻ Klenow Fragment P680G (residues 329-928 of E. Coli DNA polymerase I). In contrast, the polymerase used in the sequencing method of Braslavsky et al., which yielded sequence information for up to a maximum of five consecutive nucleic acids discussed above, was an exo⁻ Klenow Fragment. Klenow Fragment is an N-terminal truncation of E. coli DNA Polymerase I which retains both polymerase activity and 3′→5′ exonuclease activity, but has lost the 5′→3′ exonuclease activity. Exo⁻ Klenow Fragment has a mutation (D355A, E357A) at the 3′→5′ exonuclease active site which abolishes the 3′→5′ exonuclease activity of the wild type Klenow fragment, and thus has no exonuclease activity in either direction.

Tuske et al. (2000) describe a further modification of this Exo⁻ Klenow Fragment wherein the proline at position 680 was replaced with a glycine (P680G) (SEQ ID NO: 4), and show that this P680G mutation reduces template directed DNA synthesis by the P680G Exo⁻ Klenow Fragment because it dissociates from the DNA after each individual nucleotide incorporation. Tuske et al. measured the processivity of the P860G Exo⁻ Klenow fragment in a time course incorporation of dTTP on a template of poly(dA)(dT)₁₈ and found that the P680G Exo⁻ Klenow Fragment is able to catalyze addition of only a single nucleotide onto the primer strand, thus characterizing the P860G mutated Exo⁻ Klenow fragment as a non-processive polymerase.

Provided herein are methods of sequencing a single polynucleotide molecule comprising the mutant Klenow exo⁻ polymerase having a P680G mutation, which take advantage of the mutant polymerase's property of limited processivity, such that a single labeled nucleotide is added in a template dependent manner to the growing strand that is complementary to the polynucleotide template strand. The detection and analysis of a first newly added, labeled, single nucleotide before the addition of a second newly added, labeled, single nucleotide allow for improved accuracy and sensitivity in sequencing.

Labeled nucleotides: Labeled nucleotides of the invention include any nucleotide that has been modified to include a label that is directly or indirectly detectable. Such labels include optically-detectable labels such fluorescent labels, including fluorescein, rhodamine, phosphor, polymethadine dye, fluorescent phosphoramidite, texas red, green fluorescent protein, acridine, cyanine, cyanine 5 dye, cyanine 3 dye, 5-(2′-aminoethyl)-aminonaphthalene-1-sulfonic acid (EDANS), BODIPY, ALEXA, or a derivative or modification of any of the foregoing. The terms “fluorophore” and “fluorescent reporter group” are intended to include any compound, label, or moiety that absorbs energy, typically from an illumination source, to reach an electronically excited state, and then emits energy, typically at a characteristic wavelength, to achieve a lower energy state. For example but without limitation, when certain fluorophores are illuminated by an energy source with an appropriate excitation wavelength, typically an incandescent or laser light source, photons are emitted at a characteristic fluorescent emission wavelength by the fluorophore. Fluorophores, sometimes referred to as fluorescent dyes, may typically be divided into families, such as fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Exemplary fluorophores include indocarbocyanine (C3), indodicarbocyanine (C5), Cy3, Cy3.5, Cy5, Cy5.5, Cy7, Texas Red, Pacific Blue, Oregon Green 488, Alexa Fluor 488, Alexa Fluor 532, Alexa Fluor 546, Alexa Fluor 568, Alexa Fluor 594, Alexa Fluor 647, Alexa Fluor 660, Alexa Fluor 680, JOE, Lissamine, Rhodamine Green, BODIPY, fluorescein isothiocyanate (FITC), carboxy-fluorescein (FAM), phycoerythrin, rhodamine, dichlororhodamine (dRhodaminew), carboxy tetramethylrhodamine (TAMRAw), carboxy-X rhodamine (ROXm), LIZ, VIC, NED, PET, SYBR, PicoGreen, RiboGreen, and the like. Descriptions of fluorophores and their use can be found in, among other places, R. Haugland, Handbook of Fluorescent Probes and Research Products, gth ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va. Near-infrared dyes are expressly within the intended meaning of the terms fluorophore and fluorescent reporter group.

In certain instances where the fluorophore is attached to the nucleotide base, it may be equipped with a fluorophore of a relatively large size, such as fluorescein. However, smaller fluorophores, e.g., pyrene or dyes from the coumarin family, could prove advantageous in terms of being tolerated to a larger extent by polymerases. Fluorescent labels can be attached to nucleotides at a variety of locations. Attachment can be made either with or without a bridging linker to the nucleotide. Nucleotide analogs for labeling of nucleic acid with fluorophores may have the fluorescent moiety attached to the nucleotide base. It can also be attached to a sugar moiety (e.g., deoxyribose) or the alpha phosphate. Attachment to the alpha phosphate might prove advantageous because this kind of linkage leaves the internal structure of the nucleic acid intact, whereas fluorophores attached to the base have been observed to distort the double helix of the synthesized molecule and subsequently inhibit further polymerase activity. See Zhu et al., “Directly Labeled DNA Probes Using Fluorescent Nucleotides with Different Length Linkers,” Nucleic Acids Res. 22: 3418 3422 (1994), and Doublie et al., “Crystal Structure of a Bacteriophage T7 DNA Replication Complex at 2.2 angstrom Resolution,” Nature 391:251.258 (1998), which are hereby incorporated by reference. Thus, thiol-group-containing nucleotides, which have been used (in the form of NTPs) for cross-linking studies on RNA polymerase, could serve as primary backbone molecules for the attachment of suitable linkers and fluorescent labels. See Hanna et al., “Synthesis and Characterization of a New Photo-Cross-Linking CTP Analog and Its Use in Photoaffinity-Labeling Escherichia-coli and T7-RNA Polymerases,” Nucleic Acids Res. 21:2073 2079 (1993), which is hereby incorporated by reference.

In an alternative embodiment of the methods described herein, the labeled nucleotides have labels linked to the nucleotide through linkers or other means that permit the label to be easily removed from the nucleotide. The labeled nucleotides may be cleaved by methods such as chemical, oxidation, reduction, acid-labile, base labile, enzymatic, electrochemical, heat and photolabile methods. Where the labels are attached by cleavable linkers, e.g., photocleavable linkers, irradiation can be used in the methods described herein to cleave the newly added labeled nucleotide after it has been detected, and before addition of the next nucleotide to the growing complement strand. In one embodiment, a label which is attached to a nucleotide through a photocleavable linker or other photochemical attachment means, is cleavable by electromagnetic energy of between about 200 and 1000 nm wavelength. Examples of commercial sources of instruments for photochemical cleavage are Aura Industries Inc. (Staten Island, N.Y.) and Agrenetics (Wilmington, Mass.). Cleavage of the linkers results in liberation of a primary amide on the tag. Examples of photocleavable linkers include nitrophenyl glycine esters, exo- and endo-2-benzonorborneyl chlorides and methane sulfonates, and 3-amino-3(2-nitrophenyl)propionic acid. Examples of enzymatic cleavage include esterases which will cleave ester bonds, nucleases which will cleave phosphodiester bonds, and proteases which cleave peptide bonds.

As described above, the nucleotides may be selected from the common Watson-Crick bases, adenine, thymine, cytosine, guanine, and uracil, or may encompass modifications of those bases, such as peptide nucleic acids, ribonucleotides, or nucleotides modified to incorporate a detectable label (e.g., with linkers or adapters), and includes any functional analog of a nucleotide.

### Methods

The contacting step of the methods described herein, in one embodiment, make use of the same label for each of the nucleotides to be added sequentially to the polynucleotide template-primer complex in the presence of Klenow exo⁻ polymerase having a P680G mutation, until a signal is detected indicating template dependent incorporation of the labeled nucleotide added at that time interval into the growing strand. Alternatively each nucleotide can have a unique label. In another embodiment, a mixture of two or more nucleotides that each have a unique label that allows them to be identified according to which base (e.g., A,C, G and T/U) each labeled nucleotide is complementary, is added at the same time in the presence of Klenow exo⁻ polymerase having a P680G mutation to the polynucleotide template-primer complex. In such an embodiment, the identity of the incorporated nucleotide is determined through discrimination of which label is incorporated. Methods of using polymerases to synthesize nucleic acids from labeled nucleotides are known. (See, e.g., U.S. Pat. Nos. 4,962,037; 5,405,747; 6,136,543; 6,210,896). Exemplary conditions used to carry out the polymerization reaction include incubating 200 uM labeled nucleotide at 25° C. in a buffer containing 50 mM tris-HCI, pH 7.8. 1 mMDTT, 5 nM polynucleotide template/primer complex, 0.01% BSA and 5 mM MgCl₂, in the presence of a polymerase, preferably of Klenow exo⁻ polymerase having a P680G mutation. Further, Anderson et al. (2005) (Biotechniques 38(2):257-263) teaches that the incorporation of fluorescently labeled nucleotides into DNA by DNA polymerases varies according to the polymerase.

As discussed above, detection and identification of the incorporated labeled nucleotide by means of its label in the growing complement strand allows the sequence of the polynucleotide template strand to be determined, one base at a time. Detection of the incorporated labeled nucleotide can be accomplished by any means suitable for detecting the label on the incorporated nucleotide, including optical detection. Detection of single molecule FRET signal reveals sequence information and facilitates interpretation of the sequencing data. Detection of FRET signal in the methods described herein can be performed accordingly to various methods described in the art (e.g., U.S. Pat. No. 5,776,782) In some embodiments, fluorescent excitation is exerted with a Q-switched frequency doubled Nd YAG laser, which has a KHz repetition rate, allowing many samples to be taken per second. For example, a wavelength of 532 nm is useful for the excitation of rhodamine and has been used with single molecule detection schemes (Smith et al., Science 253:1122, 1992). A pulsed laser allows time resolved experiments, which are useful for rejecting extraneous noise. In some methods, excitation can be performed with a mercury lamp and signals from the incorporated nucleotides can be detected with an CCD camera (see, e.g., Unger et al., Biotechniques 27:1008, 1999).

Incorporated signals can be detected by scanning the substrates. The substrates can be scanned simultaneously or serially, depending on the scanning method used. The signals can be scanned using a CCD camera (TE/CCD512SF, Princeton Instruments, Trenton, N.J.) with suitable optics (Ploem, J. S., in Fluorescent and Luminescent Probes for Biological Activity, Mason, T. W., Ed., Academic Press, London, pp. 1-11, 1993), such as described in Yershov et al. (Proc. Natl. Acad. Sci. 93:4913, 1996), or can be imaged by TV monitoring (Khrapko et al., DNA Sequencing 1:375, 1991). The scanning system should be able to reproducibly scan the substrates. Where appropriate, e.g., for a two dimensional substrate where the substrates are localized to positions thereon, the scanning system should positionally define the substrates attached thereon to a reproducible coordinate system. The positional identification of substrates should be repeatable in successive scan steps.

Various scanning systems can be employed in the methods and apparatus of the present invention. For example, electro-optical scanning devices described in, e.g., U.S. Pat. No. 5,143,854, are suitable for use with the present invention. The system could exhibit many of the features of photographic scanners, digitizers or even compact disk reading devices. For example, a model no. PM500-A1 x-y translation table manufactured by Newport Corporation can be attached to a detector unit. The x-y translation table is connected to and controlled by an appropriately programmed digital computer such as an IBM PC/AT or AT compatible computer. The detection system can be a model no. R943-02 photomultiplier tube manufactured by Hamamatsu, attached to a preamplifier, e.g., a model no. SR440 manufactured by Stanford Research Systems, and to a photon counter, e.g., an SR430 manufactured by Stanford Research System, or a multichannel detection device. Although a digital signal are usually preferred, there can be circumstances where analog signals would be advantageous and would be known by one of skill in the art when one or the other is useful.

The stability and reproducibility of the positional localization in scanning determine, to a large extent, the resolution for separating closely positioned polynucleotide clusters on a two dimensional substrate. As the successive monitoring at a given position depends upon the ability to map the results of a reaction cycle to its effect on a positionally mapped polynucleotide, high resolution scanning is preferred. As the resolution increases, the upper limit to the number of possible polynucleotides which can be sequenced on a single matrix also increases. The limitations on the resolution can be diffraction limited and advantages can arise from using shorter wavelength radiation for fluorescent scanning steps. However, with increased resolution, the time required to fully scan a matrix can increased and a compromise between speed and resolution can be selected. Parallel detection devices which provide high resolution with shorter scan times are applicable where multiple detectors are moved in parallel.

In some applications, resolution often is not so important and sensitivity is emphasized. However, the reliability of a signal can be pre-selected by counting photons and continuing to count for a longer period at positions where intensity of signal is lower. Although this may decrease scan speed, it can increase reliability of the signal determination. Various signal detection and processing algorithms can be incorporated into the detection system. In some methods, the distribution of signal intensities of pixels across the region of signal are evaluated to determine whether the distribution of intensities corresponds to a time positive signal.

After detection and identification of an incorporated labeled nucleotide into the growing complement strand, and before the incorporation of another labeled nucleotide into the growing complement strand, the label of the incorporated nucleotide is rendered undetectable by removing the label from the nucleotide and/or extended primer, or neutralizing the label, or masking the label. In certain embodiments of the methods described herein provide for neutralizing a label by photobleaching. This is accomplished, for example, by focusing a laser with a short laser pulse, for example, for a short duration of time with increasing laser intensity. In other embodiments, a label is removed from its nucleotide by photocleavage. For example, a light-sensitive label bound to a nucleotide is photocleaved by focusing a particular wavelength of light on the label. Generally, it may be preferable to use lasers having differing wavelengths for exciting and photocleaving. Labels also can be chemically cleaved. Labels may be removed from a substrate using reagents, such as NaOH, dithiothreitol, or other appropriate buffer reagent. The use of disulfide linkers to attach the label to the nucleotide are especially useful and are known in the art. Optionally, in some embodiments the sample comprising the complex of the polynucleotide complex and its growing complement strand is washed. Optionally, in some embodiments of the methods described herein, the cycle of incorporating a labeled nucleotide into the growing complement strand in the presence of P=>G mutated exo⁻ Klenow Fragment and detecting and identifying the incorporated labeled nucleotide, are repeated one or more times, and in some embodiments, repeated until the entire sequence of the polynucleotide template is determined.

In one embodiment of the methods described herein, a combination of evanescent wave microscopy and single-pair fluorescence resonance energy transfer (spFRET; refs. 24-2624. Weiss, S. (1999) Science 283, 1676-1683, Ha, T. (2001) Methods 25, 78-86, Ha, T. J., Ting, A. Y., Liang, J., Caldwell, W. B., Deniz, A. A., Chemla, D. S., Schultz, P. G. & Weiss, S. (1999) Proc. Natl. Acad. Sci. USA 96, 893-898.) is used to reduce unwanted noise, resulting in part from repeated washings of the polynucleotide template-primer complex.

In certain embodiments of the invention, a template polynucleotide may be identified or sequenced using fluorescence resonance energy transfer (FRET). FRET is a spectroscopic phenomenon used to detect proximity between fluorescent donor and acceptor molecules. The donor and acceptor pairs are chosen such that fluorescent emission from the donor overlaps the excitation spectrum of the acceptor. When the two molecules are associated at a distance of less than 100 Angstroms, the excited-state energy of the donor is transferred non-radioactively to the acceptor and the donor emission is quenched. If the acceptor molecule is a fluorophore then its emission is enhanced. Compositions and methods for use of FRET with oligonucleotides are known (e.g., U.S. Pat. No. 5,866,366). Since FRET only reaches very short distances including about 20 nucleotides and decays at the reciprocal sixth power of distance, the excited donor molecule transfers its energy only to nearby acceptor fluorophores, which emit the spectrally resolved acceptor fluorescence of each labeled nucleotide as it is added. Distance and orientation constraints of energy transfer reduce the effective range of observation to less than 60 angstroms, thereby effectively eliminating background fluorescence from unincorporated nucleotides.

Fluorescence resonance energy transfer in the context of sequencing is described generally in Braslavsky, et al., Sequence Information can be Obtained from Single DNA Molecules, Proc. Nat'l Acad. Sci., 100: 3960-3964 (2003), incorporated by reference herein. Essentially, in one embodiment, single-pair fluorescence resonance energy transfer (spFRET) a donor fluorophore is attached to the primer, polymerase, or template, as well as the labeled nucleotide(s). Nucleotides added for incorporation into the primer comprise an acceptor fluorophore that is activated by the donor when the two are in proximity. Activation of the acceptor causes it to emit a characteristic wavelength of light. In this way, incorporation of a nucleotide in the primer sequence is detected by detection of acceptor emission. Spectroscopically, when the donor is excited, its specific emission intensity decreases while the acceptor's specific emission intensity increases, resulting in fluorescence enhancement.

In some embodiments of the methods described herein, incorporation of different types of nucleotides into a primer is detected using different fluorescent labels on the different types of nucleotides. When two different labels are incorporated into the primer in close vicinity, signals due to fluorescence resonance energy transfer (FRET) can be detected. FRET is a phenomenon that has been well documented in the literature, e.g., in T. Foster, Modem Quantum Chemistry, Istanbul Lectures, Part III, 93-137, 1965, Academic Press, New York; and Selvin, “Fluorescence Resonance Energy. Transfer,”. Methods in Enzymology 246: 300-335, 1995. In FRET, one of the fluorophores (donor) has an emission spectrum that overlaps the excitation spectrum of the other fluorophore (acceptor) and transfer of energy takes place from the donor to the acceptor through fluorescence resonance energy transfer. The energy transfer is mediated by dipole-dipole interaction. Spectroscopically, when the donor is excited, its specific emission intensity decreases while the acceptor's specific emission intensity increases, resulting in fluorescence enhancement.

Any of a number of fluorophore combinations can be selected for labeling the nucleotides in the present invention for detection of FRET signals (see for example, Pesce et al., eds, Fluorescence Spectroscopy, Marcel Dekker, New York, 1971; White et al., Fluorescence Analysis: A practical Approach, Marcel Dekker, New York, 1970; Handbook of Fluorescent Probes and Research Chemicals, 6th Ed, Molecular Probes, Inc., Eugene, Oreg., 1996; which are incorporated by reference). In general, a preferred donor fluorophore is selected that has a substantial spectrum of the acceptor fluorophore. Furthermore, it may also be desirable in certain applications that the donor have an excitation maximum near a laser frequency such as Helium-Cadmium 442 nm or Argon 488 nm. In such applications the use of intense laser light can serve as an effective means to excite the donor fluorophore. The acceptor fluorophore has a substantial overlap of its excitation spectrum with the emission spectrum of the donor fluorophore. In addition, the wavelength of the maximum of the emission spectrum of the acceptor moiety is preferably at least 10 nm greater than the wavelength of the maximum of the excitation spectrum of the donor moiety. The emission spectrum of the acceptor fluorophore is shifted compared to the donor spectrum.

Suitable donors and acceptors operating on the principle of fluorescence energy transfer (FET) include, but are not limited to, 4-acetamido-4′-isothiocyanatostilbene-2,2′disulfonic acid; acridine and derivatives: acridine, acridine isothiocyanate; 5-(2′-aminoethyl)aminonap-hthalene-1-sulfonic acid (EDANS); 4-amino-N-[3-vinylsulfonyl)phenyl]naphth-alimide-3,5disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120), 7-amino-4-trifluor-omethylcouluarin (Coumaran 151); cyanine dyes; cyanosine; 4′,6-diaminidino-2-phenylindole (DAPI); 5′,5″-dibromopyrogallol-sulfonap-hthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-fluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron.™. Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolla Blue; phthalo cyanine; and naphthalo cyanine.

In some embodiments, the FRET donor or the FRET acceptor may be on the polymerase. In certain embodiment, the polymerase is attached to a surface.

In some embodiments, fluorescent excitation is exerted with a Q-switched frequency doubled Nd YAG laser, which has a KHz repetition rate, allowing many samples to be taken per second. For example, a wavelength of 532 nm is useful for the excitation of rhodamine and has been used with single molecule detection schemes (Smith et al., Science 253:1122, 1992). A pulsed laser allows time resolved experiments, which are useful for rejecting extraneous noise. In some methods, excitation can be performed with a mercury lamp and signals from the incorporated nucleotides can be detected with an CCD camera (see, e.g., Unger et al., Biotechniques 27:1008, 1999).

Incorporated signals can be detected by scanning the substrates. The substrates can be scanned simultaneously or serially, depending on the scanning method used. The signals can be scanned using a CCD camera (TE/CCD512SF, Princeton Instruments, Trenton, N.J.) with suitable optics (Ploem, J. S., in Fluorescent and Luminescent Probes for Biological Activity, Mason, T. W., Ed., Academic Press, London, pp. 1-11, 1993), such as described in Yershov et al. (Proc. Natl. Acad. Sci. 93:4913, 1996), or can be imaged by TV monitoring (Khrapko et al., DNA Sequencing 1:375, 1991). The scanning system should be able to reproducibly scan the substrates. Where appropriate, e.g., for a two dimensional substrate where the substrates are localized to positions thereon, the scanning system should positionally define the substrates attached thereon to a reproducible coordinate system. The positional identification of substrates should be repeatable in successive scan steps.

Various scanning systems can be employed in the methods and apparatus of the present invention. For example, electro-optical scanning devices described in, e.g., U.S. Pat. No. 5,143,854, are suitable for use with the present invention. The system could exhibit many of the features of photographic scanners, digitizers or even compact disk reading devices. For example, a model no. PM500-A1 x-y translation table manufactured by Newport Corporation can be attached to a detector unit. The x-y translation table is connected to and controlled by an appropriately programmed digital computer such as an IBM PC/AT or AT compatible computer. The detection system can be a model no. R943-02 photomultiplier tube manufactured by Hamamatsu, attached to a preamplifier, e.g., a model no. SR440 manufactured by Stanford Research Systems, and to a photon counter, e.g., an SR430 manufactured by Stanford Research System, or a multichannel detection device. Although a digital signal are usually preferred, there can be circumstances where analog signals would be advantageous and would be known by one of skill in the art when one or the other is useful.

The stability and reproducibility of the positional localization in scanning determine, to a large extent, the resolution for separating closely positioned polynucleotide clusters on a two dimensional substrate. As the successive monitoring at a given position depends upon the ability to map the results of a reaction cycle to its effect on a positionally mapped polynucleotide, high resolution scanning is preferred. As the resolution increases, the upper limit to the number of possible polynucleotides which can be sequenced on a single matrix also increases. The limitations on the resolution can be diffraction limited and advantages can arise from using shorter wavelength radiation for fluorescent scanning steps. However, with increased resolution, the time required to fully scan a matrix can increased and a compromise between speed and resolution can be selected. Parallel detection devices which provide high resolution with shorter scan times are applicable where multiple detectors are moved in parallel.

In some applications, resolution often is not so important and sensitivity is emphasized. However, the reliability of a signal can be pre-selected by counting photons and continuing to count for a longer period at positions where intensity of signal is lower. Although this may decrease scan speed, it can increase reliability of the signal determination. Various signal detection and processing algorithms can be incorporated into the detection system. In some methods, the distribution of signal intensities of pixels across the region of signal are evaluated to determine whether the distribution of intensities corresponds to a time positive signal.

After detection and identification of an incorporated labeled nucleotide into the growing complement strand, and before the incorporation of another labeled nucleotide into the growing complement strand, the label of the incorporated nucleotide is rendered undetectable by removing the label from the nucleotide and/or extended primer, or neutralizing the label, or masking the label. In certain embodiments of the methods described herein provide for neutralizing a label by photobleaching. This is accomplished, for example, by focusing a laser with a short laser pulse, for example, for a short duration of time with increasing laser intensity. In other embodiments, a label is removed from its nucleotide by photocleavage. For example, a light-sensitive label bound to a nucleotide is photocleaved by focusing a particular wavelength of light on the label. Generally, it may be preferable to use lasers having differing wavelengths for exciting and photocleaving. Labels also can be chemically cleaved. Labels may be removed from a substrate using reagents, such as NaOH, dithiothreitol, or other appropriate buffer reagent. The use of disulfide linkers to attach the label to the nucleotide are especially useful and are known in the art. Optionally, in some embodiments the sample comprising the complex of the polynucleotide complex and its growing complement strand is washed. Optionally, in some embodiments of the methods described herein, the cycle of incorporating a labeled nucleotide into the growing complement strand in the presence of P=>G mutated exo⁻ Klenow Fragment and detecting and identifying the incorporated labeled nucleotide, are repeated one or more times, and in some embodiments, repeated until the entire sequence of the polynucleotide template is determined.

### EXAMPLE 1

Approximately 20 pmol of template DNA was polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62). The average dA tail length was 50±5 nucleotides. Terminal transferase was then used to label the polyadenylated templates with Cy3-dUTP. Polyadenylated labeled tempaltes were then terminated with dideoxyTTP (also added using terminal transferase). The resulting templates were filtered with a YM10 ultrafiltration spin column to remove free nucleotides and stored in ddH₂O at −20° C.

Epoxide-coated glass slides were prepared for oligo attachment. Epoxide-functionalized 40 mm diameter #1.5 glass cover slips (slides) were obtained from Erie Scientific (Salem, N.H.). The slides were preconditioned by soaking in 3×SSC for 15 minutes at 37° C. Next, a 500 pM aliquot of 5′ aminated templates described above were incubated with each slide for 30 minutes at room temperature in a volume of 80 ml. The resulting slides have poly(dA50) templates attached by direct amine linkage to the epoxide. The slides are then treated with phosphate (1 M) for 4 hours at room temperature in order to passivate the surface. Slides re then stored in polymerase rinse buffer (20 mM Tris, 100 mM NaCl, 0.001% Triton X-100, pH 8.0) until they are used for sequencing.

For sequencing, the slides were placed in a modified FCS2 flow cell (Bioptechs, Butler, Pa.) using a 50 um thick gasket The flow cell was placed on a movable stage that is part of a high-efficiency fluorescence imaging system built around a Nikon TE-2000 inverted microscope equipped with a total internal reflection (TIR) objective. The slide was then rinsed with HEPES buffer with 100 mM NaCl and equilibrated to a temperature of 50° C. A 1 nM aliquot of poly(dT50) primer in 3×SSC was placed in the flow cell and incubated on the slide for 20 minutes. After incubation, the flow cell was rinsed with 1×SSC/HEPES/0.1% SDS followed by HEPES/NaCl. A passive vacuum apparatus was used to pull fluid across the flow cell. The resulting slide contained template/oligo(dT) primer duplex. The temperature of the flow cell was then reduced to 37° C. for sequencing and the objective was brought into contact with the flow cell.

For sequencing, cytosine triphosphate, guanidine triphosphate, adenine triphosphate, and uracil triphosphate, each having a cyanine-5 label (at the 7-deaza position for ATP and GTP and at the C5 position for CTP and UTP (PerkinElmer)) were stored separately in buffer containing 20 mM Tris-HCl, pH 8.8, 10 mM MgSO₄, 10 mM (NH₄)₂SO₄, 10 mM HCl, and 0.1% Triton X-100, and 100 U P680G polymerase (NEN). Sequencing proceeds as follows.

First, initial imaging was used to determine the positions of duplex on the epoxide surface. The Cy3 label attached to the templates was imaged by excitation using a laser tuned to 532 nm radiation (Verdi V-2 Laser, Coherent, Inc., Santa Clara, Calif.) in order to establish duplex position. For each slide only single fluorescent molecules imaged in this step were counted. Imaging of incorporated nucleotides as described below was accomplished by excitation of a cyanine-5 dye using a 635 nm radiation laser (Coherent). 250 nM Cy5CTP was placed into the flow cell and exposed to the slide for 2 minutes. After incubation, the slide was rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes). An oxygen scavenger containing 30% acetonitrile and scavenger buffer (134 ul HEPES/NaCl, 24 ul 100 mM Trolox in MES, pH6.1, 10 ul DABCO in MES, pH6.1, 8 ul 2M glucose, 20 ul Nal (50 mM stock in water), and 4 ul glucose oxidase) was next added. The slide was then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence were recorded. After imaging, the flow cell was rinsed 5 times each with SSC/HEPES/SDS (60 ul) and HEPES/NaCl (60 ul). Next, the cyanine-5 label was cleaved off incorporated CTP by introduction into the flow cell of 50 mM TCEP for 5 minutes, after which the flow cell was rinsed 5 times each with SSC/HEPES/SDS (60 ul) and HEPES/NaCl (60 ul). The remaining nucleotide was capped with 50 mM iodoacetamide for 5 minutes followed by rinsing 5 times each with SSC/HEPES/SDS (60 ul) and HEPES/NaCl (60 ul). The scavenger was applied again in the manner described above, and the slide was again imaged to determine the effectiveness of the cleave/cap steps and to identify non-incorporated fluorescent objects.

The procedure described above was then conducted 500 nM Cy5dUTP, followed by 250 nM Cy5dGTP, and finally 500 nM Cy5dATP. The procedure (expose to nucleotide, polymerase, rinse, scavenger, image, rinse, cleave, rinse, cap, rinse, scavenger, final image) is repeated exactly as described for ATP, GTP, and UTPs. Uridine was used instead of Thymidine due to the fact that the Cy5 label was incorporated at the position normally occupied by the methyl group in Thymidine triphosphate, thus turning the dTTP into dUTP. In all 12 cycles (C, U, A, G) were conducted as described in this and the preceding paragraph.

Once the desired number of cycles were completed, the image stack data (i.e., the single molecule sequences obtained from the various surface-bound duplex) was analyzed and compared to the known template sequence.

The contents of all references, patents and patent applications (including, published patent applications) cited throughout this application are hereby incorporated by reference.

What is claimed is:

1. A method for determining the sequence of a nucleic acid comprising the steps of;
contacting a nucleic acid duplex, comprising primer nucleic acid hybridized to a template nucleic acid with a Klenow exo⁻ DNA polymerase having a glycine for proline substitution at position 680 in the presence of a first labeled nucleotide under conditions that permit the polymerase to add nucleotides to said primer in a template-dependent manner, detecting a signal from the incorporated labeled nucleotide, and repeating said contacting and detecting steps at least once, wherein sequential detection of incorporated labeled nucleotide determines the sequence of the nucleic acid.

2. The method of claim 1, wherein said duplex is attached to a surface.

3. The method of claim 2, wherein said surface comprises a plurality of duplex immobilized at different positions on the substrate.

4. The method of claim 3, wherein at least some of said duplex are individually optically resolvable.

5. The method of claim 1, wherein said label is a fluorescent label.

6. The method of claim 5, wherein the fluorescent label comprises one or more of Cy3 or Cy4.

7. The method of claim 1, wherein the presence or absence of label is determined with total internal reflection fluorescence (TIRF) microscopy.

8. A Klenow exo⁻ DNA polymerase for single molecule sequencing comprising a glycine for proline substitution at position 680.
